(19) United States
(12) Patent Application Publication
(10) Pub. No.: US 2009/0281380 A1
(43) Pub. Date: Nov. 12, 2009

(54) System and method for assembling a swallowable sensing device

(76) Inventors: Shahar Miller, Givat Ada (IL); Zvika Gilad, Haifa (IL); Vitaly Fastovksy, Haifa (IL)

Correspondence Address: Pearl Cohen Zedek Latzer, LLP, 1500 Broadway, 12th Floor, New York, NY, 10036, US

(21) Appl. No.: 11/794,576

(22) PCT Filed: Jan. 1, 2006
(86) PCT No.: PCT/IL2006/000003

§ 371 Date: Jul. 2, 2007

Related U.S. Application Data
(60) Provisional application No. 60/639,986, filed on Dec. 30, 2004.

(51) Int. Cl. A61B 1/04; B29C 65/00

(52) U.S. Cl. 600/109; 156/272.8

(57) ABSTRACT

A system and method of assembling a swallowable sensor, including attaching a first piece of a shell of the sensor to a second piece of the shell of the sensor, where the attaching may include for example screwing the first piece to the second piece, welding or gluing the first piece to the second piece, snapping the first piece to the second piece or for example applying laser energy to a pigment in the first piece.

### FIELD OF THE INVENTION

The present invention relates to a system and method for assembling a swallowable sensing device.

### BACKGROUND OF THE INVENTION

Known in-vivo and/or ingestible imaging devices, e.g. ingestible imaging capsules may include a plurality of electrical components, e.g. an imager, a transmitter, batteries, an antenna, etc. that may be enclosed and sealed within a thin housing shell. The seal may prevent, for example, water and/or air from entering the housing of the in-vivo imaging capsule and/or may prevent leakage, e.g. battery leakage from leaking out of the in-vivo imaging capsule. Bonding, attaching or sealing thin materials together may generally be performed with for example glues, heat, ultra-violet curing or other processes. Certain processes are time consuming, inconvenient or inaccurate and may be unsuitable for precision bonding where delicate materials are to be sealed against water, air or other elements.

### SUMMARY OF THE INVENTION

Thus, the present invention provides, according to some embodiments an in-vivo sensing device, e.g. an in-vivo imaging device, which may include a shell and/or housing, for example a two parts shell such as a front and rear outer shell. According to one embodiment of the present invention, a connecting ring may be embedded within an in-vivo imaging device portion for example in a front shell, enabling the joining and attachment of two in-vivo imaging device components, such as the front and rear shells.

According to some embodiments of the present invention, the in-vivo imaging device components may be glued together, for example according to one embodiment the rear shell may be glued to a connecting ring embedded within a front shell.

According to embodiments of the present invention, the in-vivo imaging device components may be joined together by friction fitting, press fitting, snap fitting, laser welding, laser melting, spin welding, and/or ultrasonic welding.

According to an embodiment of the present invention, the various sensing device components may be attached and/or sealed and/or waterproofed by inserting the components into a molding cast and pouring the surrounding housing material into the mold.

### BRIEF DESCRIPTION OF THE DRAWINGS

The principles and operation of the system, apparatus, and method according to the present invention may be better understood with reference to the drawings, and the following description, it being understood that these drawings are given for illustrative purposes only and are not meant to be limiting, wherein:

FIG. 1 schematically illustrates an in-vivo sensing device according to one embodiment of the present invention;

FIGS. 2A-2B schematically illustrates possible joining and sealing of two shells according to embodiments of the present invention;

FIGS. 3A-3C schematically illustrates possible joining and sealing of shells according to embodiments of the present invention;

FIG. 3D shows a schematic flow-chart of a method for attaching components of an in-vivo device, according to embodiment of the present invention;

FIGS. 4A-4B schematically illustrate a close-up of two connecting rings, according to some embodiments of the present invention;

FIG. 4C shows a schematic flow-chart of a method for attaching components of an in-vivo device, according to some embodiments of the present invention;

FIG. 5A schematically illustrates a close-up of two connecting parts, according to an embodiment of the present invention;

FIG. 5B schematically illustrates an in-vivo imaging device according to an embodiment of the present invention;

FIG. 5C shows a schematic flow-chart of a method for attaching components of an in-vivo device, according to embodiments of the present invention;

FIG. 6A depicts a casting system for attaching components of an in-vivo sensing device 40, in accordance with an embodiment of the invention;

FIG. 6B shows a schematic flow-chart of a method for attaching components of an in-vivo device, according to another embodiment of the present invention; and

FIG. 7 shows a schematic diagram of a laser sealing two thin materials in accordance with an embodiment of the invention.

It should be noted that for simplicity and clarity of illustration, elements shown in the figures have not necessarily been drawn to scale. For example, the dimensions of some of the elements may be exaggerated relative to other elements for clarity. Furthermore, where considered appropriate, reference numerals may be repeated among the figures to indicate corresponding or analogous elements throughout the serial views.

### DETAILED DESCRIPTION OF THE INVENTION

The following description is presented to enable one of ordinary skill in the art to make and use the invention as provided in the context of a particular application and its requirements. Various modifications to the described embodiments will be apparent to those with skill in the art, and the general principles defined herein may be applied to other embodiments. Therefore, the present invention is not intended to be limited to the particular embodiments shown and described, but is to be accorded the widest scope consistent with the principles and novel features herein disclosed. In the following detailed description, numerous specific details are set forth in order to provide a thorough understanding of the present invention. However, it will be understood by those skilled in the art that the present invention may be practiced without these specific details. In other instances, well-known methods, procedures, and components have not been described in detail so as not to obscure the present invention.

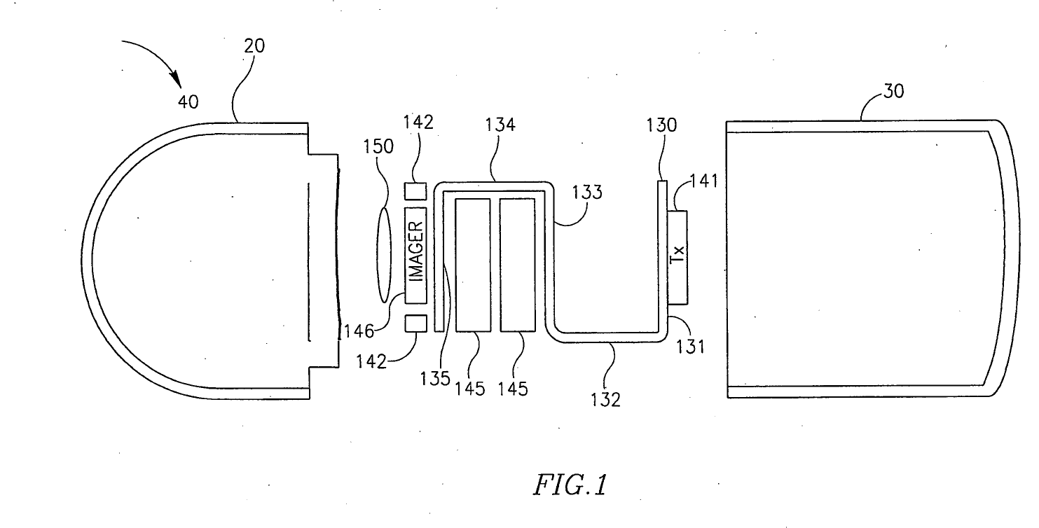

FIG. 1 depicts components of a device 40, for example an in-vivo imaging device, prior to the assembly of the device's components, for example before the insertion or assembly of the device's components into the device body, according to some embodiments of the present invention. According to one embodiment of the present invention, the device 40 may include a sensing unit, for example an imager 146, one or more illumination sources 142, one or more power sources 145, a transmitter 141, a front shell 20, for example a viewing dome, and a rear shell 30, for example a cover e.g. a transparent cover. In some embodiments, device 40 may take on a shape and size of a swallowable capsule, but other sorts of devices or suitable configurations may be used. Outside a patient's body may be, for example, an image receiver (including, for example, an antenna or an antenna array), a storage unit, a data processor, and a monitor.

According to one embodiment of the present invention, transmitter 141 may operate using radio waves; but in some embodiments, such as those where device 40 is or is included within an endoscope, transmitter 141 may transmit data via, for example, wire, optical fiber and/or other suitable methods. Other suitable methods or components for wired or wireless transmission may be used.

Device 40 typically may be or may include an autonomous swallowable capsule, but device 40 may have other shapes and need not be swallowable or autonomous. Embodiments of device 40 are typically autonomous, and are typically self-contained. For example, device 40 may be a capsule or other unit where all the components are substantially contained within a container or shell, and where device 40 does not require any wires or cables to, for example, receive power or transmit information.

In one embodiment, device 40 may include an in-vivo video camera, for example, an imager 146, which may capture and transmit images of, for example, the gastrointestinal (GI) tract while device 40 passes through the GI lumen. Other lumens and/or body cavities may be imaged and/or sensed by device 40. In some embodiments, imager 146 may include, for example, a Charge Coupled Device (CCD) camera or imager, a Complementary Metal Oxide Semiconductor (CMOS) camera or imager, a digital camera, a stills camera, a video camera, or other suitable imagers, cameras, or image acquisition components.

In some embodiments, device 40 may include one or more illumination sources 142, for example one or more Light Emitting Diodes (LEDs), “white LEDs”, or other suitable light sources. Illumination sources 142 may, for example, illuminate a body lumen or cavity being imaged and/or sensed. An optional optical system 150, including, for example, one or more optical elements, such as one or more lenses or composite lens assemblies, one or more suitable optical filters, or any other suitable optical elements, may optionally be included in device 40 and may aid in focusing reflected light onto imager 146 and/or performing other light processing operations.

In accordance with some embodiments of the invention, device 40 may include a circuit board 130 which may have one or more rigid portions and one or more flexible portions. For example, circuit board 130 may include rigid portions 131, 133 and 135, which may be interconnected through flexible portions 132 and 134. Other numbers, orders or combinations of rigid portions and/or flexible portions may be used.

The one or more flexible portions of circuit board 130, such as flexible portions 132 and 134, may allow bending, folding, twisting or positioning of circuit board 130 into certain shapes. For example, circuit board 130 may have a “2” shape, a “5” shape, a “6” shape, a “C” shape, or other suitable shapes.

In some embodiments, circuit board 130 may be manufactured to have an initial flat, non-twisted or non-folded shape, and may be later folded, bended, twisted or positioned in a desired shape within the shell 20 and shell 30 of device 40. For example, circuit board 130 may be folded or re-shaped upon its insertion into device 40, or before encapsulation of circuit board 130 inside device 40.

According to an embodiment of the present invention, the various components of the device 40 may be, for example disposed on a circuit board 130 and may be later connected to the front shell 20. In alternate embodiments, other arrangements for assembly of device 40 are possible.

According to some embodiments of the present invention, different joining, bonding and attachment methods may be used during assembly and sealing of device 40 assembly, for example welding methods e.g. laser welding and/or spin welding and/or Herman welding and/or vibration welding. According to some embodiments of the present invention melt down methods and/or ultrasonic joining methods and/or fraction fitting methods may be used for assembly and sealing of device 40.

According to some embodiments of the present invention, the methods of attachment and/or sealing may involve a third part or material that may serve as a connector or reaction catalyst, for example as in the case of a melt down method where another melted material is used to glue the two pieces together.

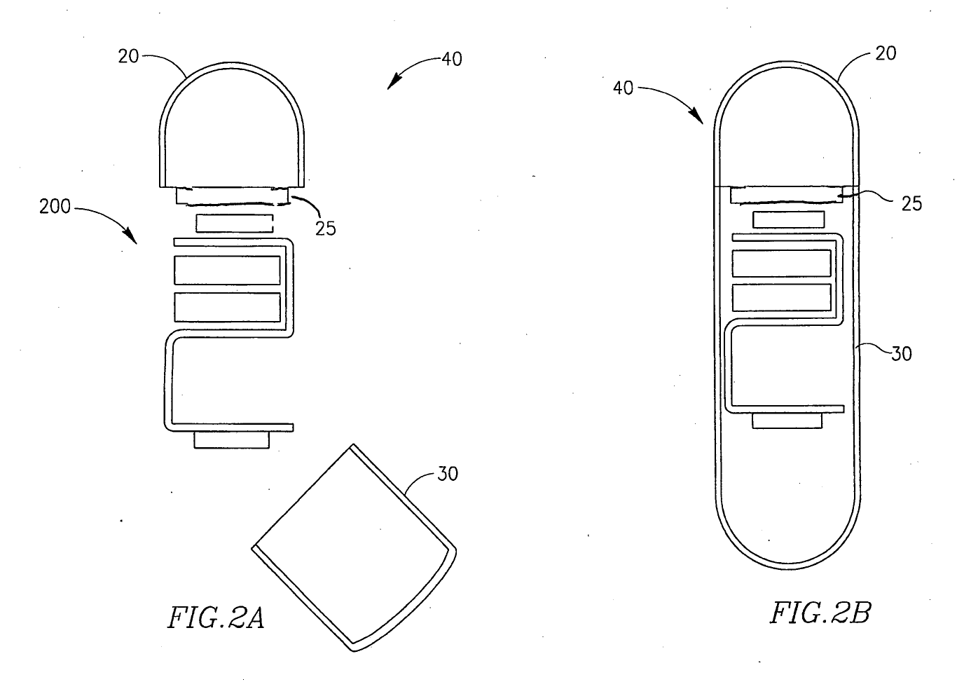

FIG. 2A schematically illustrates the attachment of device 40 components, according to one embodiment of the present invention, According to one embodiment of the invention components, for example components 200 enclosed within device 40 may be connected, for example, to the front shell 20 before the joining of the front shell 20 and the rear 30 shell.

According to some embodiments of the present invention, a connecting ring 25 such as an elastic connecting ring made, for example, from the same material and/or mold of front shell 20, may be embedded within the front shell 20 and/or assembled around a surface of front shell 20. According to one embodiment, an o-ring may be positioned around ring 25. According to one embodiment of the present invention, the front shell 20 and the rear shell 30 may have screw threads with matching grooves along its rim so that the two shells can be screwed together.

According to one embodiment of the present invention, the front shell 20 may be attached to the rear shell 30 by, for example, gluing or bonding the two parts, shell 20 and shell 30 together. According to one embodiment of the present invention, quick or slow glue, for example a UV glue, may be applied to the internal ring 25 integral to or embedded within the front shell 20 so that when the two shells 20 and 30 are brought together and arranged as desired herein, the front and rear shells 20 and 30 will be tightly glued. Other attachment methods may be used to attach shells 20 and 30 together. In addition to and/or in place of rings with screw threads, for example, friction fitting, welding, melt down etc. may be implemented for bonding shells 20 and 30 together.

FIG. 2B shows a schematic side view of the fully assembled and sealed device 40, according to some embodiments of the present invention. According to one embodiment of the present invention, the ring 25 integral or embedded within the front shell 20 may create enough pressure on the rear shell 30 when the two parts are pressed together to ensure a completely tight fitted structure, shell or housing. According to another embodiment of the present invention the ring 25 may include several sheets that create outward pressure, keeping the structure safely in place. According to another embodiment of the present invention, the ring 25 may be welded to the outer shell 20 by using a welding or meltdown method as was described in reference to FIG. 1.

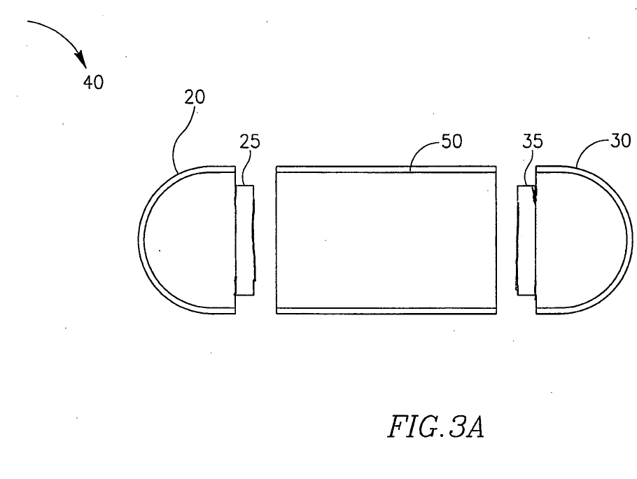

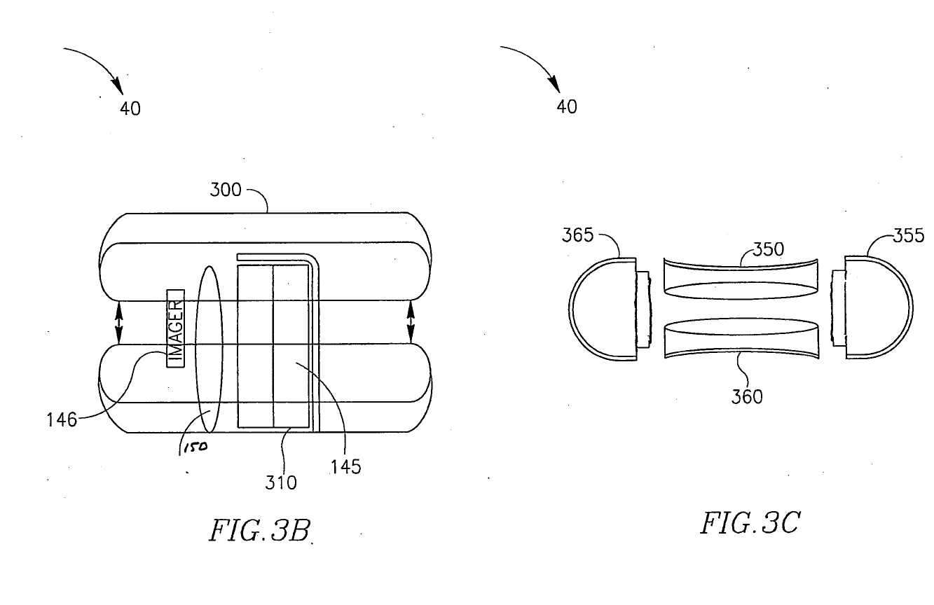

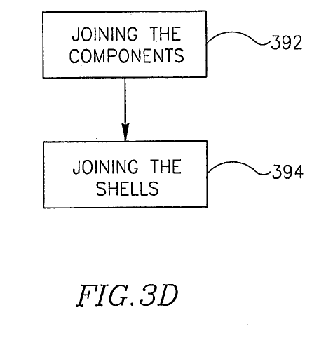

FIG. 3A schematically illustrates an assembly of the housing or/or outer shell of device 40, for example by joining three shells, according to one embodiment of the present invention. According to one embodiment of the present invention, the two front 20 and rear 30 shells may be connected or assembled onto a central shell 50, for example a cover and/or housing. According to some embodiments of the present invention, connecting rings 25 and 35 may exist to enable attachment of the front 20 and rear 30 shells, to the central compartment 50. According to one embodiment of the present invention, the connecting rings may be attached to the front and rear shells 20 and 30 with the central shell carrying, for example matching grooves or threads along its rims to enable secure screwing of the parts together. In one example, front shell 20 and rear shell 30 may be transparent and may be dome shaped. The rear front and central shells 30, 20 and 50 may be either glued or welded together, or pressure adhered according to the attachment methods as mentioned herein, for example as mentioned in FIG. 1.

FIG. 3B schematically illustrates an assembly of device 40 components, for example by joining two shells, according to some embodiments of the present invention. According to one embodiment of the present invention the device 40 may include two lengthwise or horizontal shells, for example an upper shell 300 and a bottom shell 310. The shape of the two shells, for example shells 300 and 310 may enable an in-vivo device assembly manufacturing process that may be both simple and easy to perform. For example components, such as electrical components such as cords or wiring, may be easily fitted along the length of the in-vivo device 40. According to one embodiment of the present invention, components such as the imager 146 and/or illumination source 142 and/or power source 145 may be fitted into one of the shells, for example to shell 310. According to one embodiment of the present invention, the upper shell 300 may be attached, joined or bonded to the bottom shell 310 by, for example, gluing the two shells together. Other attachment methods may be used, according to some embodiments of the present invention, to attach shells 300 and 310 together, for example, friction fitting, laser welding, melt down etc.

FIG. 3C schematically illustrates an assembly of device 40 components, for example by joining four shells, according to some embodiments of the present invention. According to one embodiment of the present invention, device 40 may include four shells, for example two lengthwise shells such as a top shell 350 and a bottom shell 360 and a front shell 365, for example a dome and rear shell 355, for example a housing cover.

A method for attaching components of an in-vivo device, for example attaching two shells, three shells, or four shells, in accordance with some embodiments of the present invention is depicted in FIG. 3D. The method for attaching components of an in-vivo device may include the following steps: joining components of device 40. For example, according to one embodiment of the present invention components enclosed within device 40, such as the imager and/or the illumination unit may be attached, for example, to one of the shells 20, 30, 50, 300, 310, 350, 360, 355, 365 of device 40, (step 392). For example, according to one embodiment of the present invention components of device 40 may be attached to the front shell 20 (as mentioned herein for example in the description of FIG. 2A) or to the top shell (as mentioned herein for example in reference to FIG. 3B); joining the shells to one another (step 394). For example as shown above with reference to in FIG. 3C shell 350 may be attached to the bottom lengthwise shell and afterwards the front shell 365 and the rear shell 355 may be attached to shells 350 and 360. According to some embodiments of the present invention the shell 355 may be adhered to one another by, for example, gluing, friction fitting, press fitting, welding, laser welding, and/or other suitable methods. Other steps may be included.

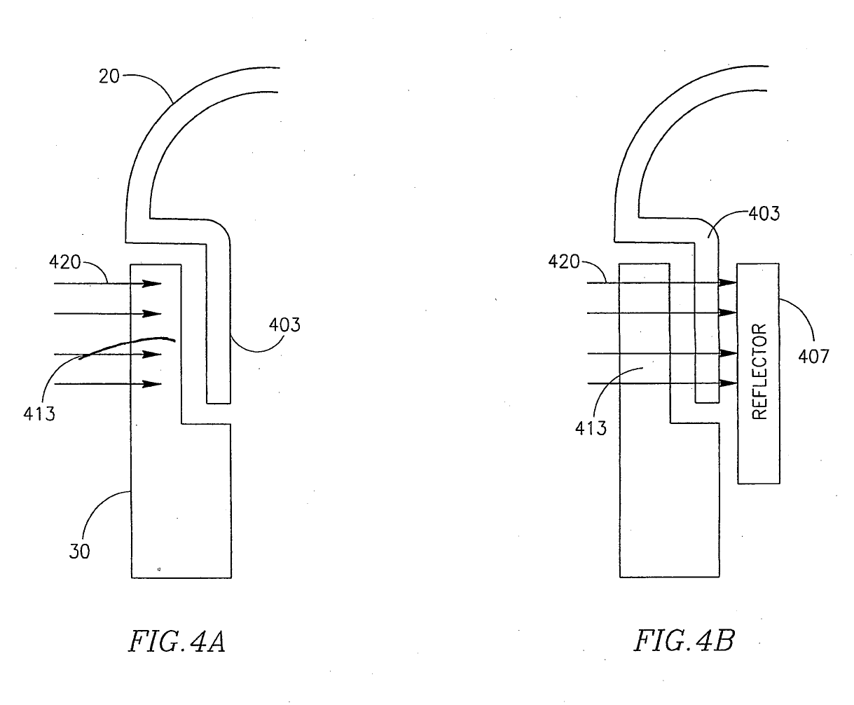

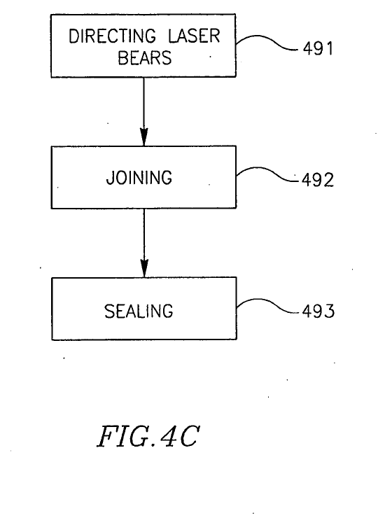

Reference is now made to FIG. 4A which schematically illustrates a close-up of two connecting rings 403 and 413, according to one embodiment of the present invention. According to some embodiments of the present invention connecting rings 403 and 413 may be used, for example to adhere one or more shells to one another, such as shells 20 and 30, for example by laser welding and/or laser melting. Laser welding and/or melting may provide a clean, precise and time conserving method for assembly of the device.

According to one embodiment of the present invention connecting ring 403 may be an extension of shell 20 and connecting ring 303 may be an extension of shell 30. In one example, shell 20 and 30 may be manufactured, for example, by injection molding and connecting ring 403 and may be made of the same material and from the same mold as shell 20, and connecting ring 413 may be made of the same material and from the same mold as shell 30. In one example, connecting ring 403 may be integral to shell 20 and connecting ring 413 may be integral to shell 30. According to some embodiments of the present invention the shells and/or connecting rings may be made out of plastic or other materials such as polycarbonate or Isoplast™.

According to one embodiment of the present invention connecting ring 413 may be made out of a material which enables a laser energy, such as laser beam(s) 420, to pass through it unobstructed, while connecting ring 403 may include an energy and/or laser absorbing material. Thus, according to one embodiment of the present invention when a laser beam is applied to the connecting rings it is absorbed in area 305 and as a result, heat is generated. The high temperature and heat generated in area 305 will bring about the melting/welding of the device shells and will join them together.

According to one embodiment of the present invention, connecting rings 413 and 403 may include energy and/or laser absorbing material. According to one embodiment of the present invention, the absorption of energy may be generated by attaching an energy absorbing part, for example a ring 409, to one of the connecting rings 403 and 413. According to one embodiment of the present invention energy absorbing material may be placed between a non-absorbing connecting rings, for example between connecting rings 403 and 413. The absorbing material is optimized for absorption in the laser beam wavelength, and may generate excessive heat in the area of the connection. The absorbing material will absorb the beam energy which will result in high temperature and heat generation which will cause melting/welding of the connecting rings.

Reference is now made to FIG. 4B which schematically illustrates a close-up of two connecting rings 403 and 413, and a reflector 407 according to one embodiment of the present invention. According to one embodiment of the present invention a reflector 407 may be attached to a connecting ring(s), for example to connecting rings 413 and/or 403, causing the laser beam(s) 420 directed to the reflector 407, to move back and forth between the connecting rings 403 and 413, and to generate heat which will cause melting/welding of the connecting parts.

According to some embodiments of the present invention, a method for attaching components of an in-vivo device by laser welding/melting, as shown in FIG. 4C, may include the following steps: directing laser beams towards a connecting rings(s), for example to a connecting ring which may include an energy absorbing material (step 491); joining one component to the other, for example joining two shells e.g. a dome and a cover component (step 492); sealing the in-vivo device (step 493). Other steps may be included and the steps may occur in a different order.

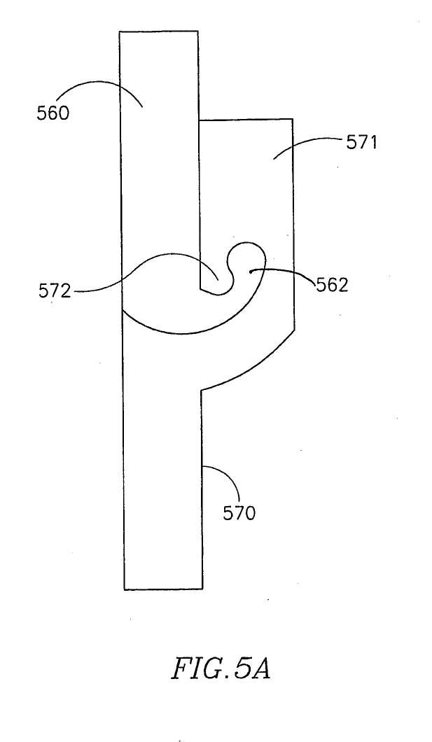

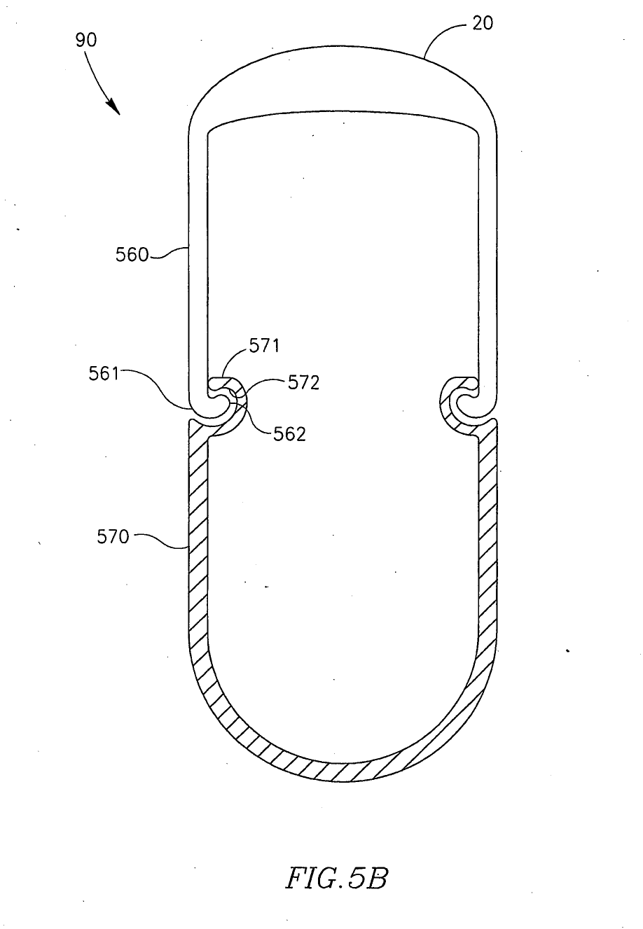

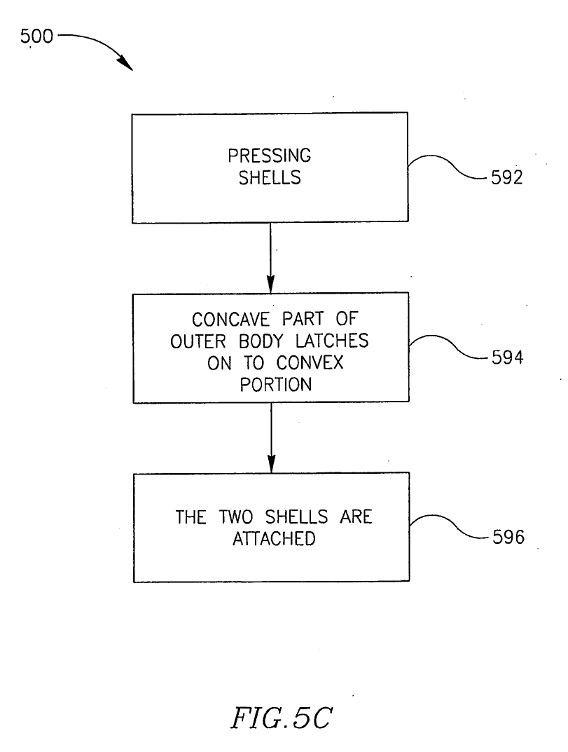

Reference is now made to FIG. 5A which schematically illustrates a close-up of two connecting rings, for example connecting rings 560 and 570, and a curve 562, which may join the two connecting rings 560 and 570, according to one embodiment of the present invention. According to one embodiment of the present invention, connecting edge 571 curves inwards, so that a concave crescent shaped protrusion 572 may be formed. A convex protrusion 562 at the end 561 of ring 560 enables the joining of connecting rings 560 and 570.

According to one embodiment of the present invention, the joining of rings 570 and 560 as depicted in FIG. 5A, may be achieved by employing a snap fitting connecting method. According to one embodiment of the present invention, a snap fitting method may include snapping and/or twisting one of the connecting rings, for example connecting ring 560 in such a way that creates high friction between the connecting rings which enables pressure fitting and sealing of the in-vivo device.

According to some embodiments of the present invention the components of the device 40, for example the shells may be adhered to one another by a friction welding method. According to one embodiment of the present invention, friction welding method may include generating heat by linearly moving in-vivo components, for example the shells 20 and 30, one against each other. The heat generated between the components may cause the melting/welding of the shells, and enables their joining.

According to some embodiments of the present invention the components of the device 40, for example the shells 20 and 30, may be adhered to one another by a spin welding method. According to the spin welding method the movement direction for example of device 40 components is circular instead of linear, e.g. one of the components is spun at relative high speed so that heat created as a result between the connecting parts may cause the melting/welding of the connecting parts.

According to some embodiments of the present invention the components of the device 40, for example the shells may be adhered to one another by a metallic substance which may be inserted between the components, for example between rings 404 and 413. According to one embodiment of the present invention, an external power supply may be applied to the metallic substance and excessive heat, which may cause the melting/welding of the connecting parts, may be thus generated between the components.

According to some embodiments of the present invention the components of the device 40, for example the shells may be adhered to one another by an ultra sonic welding method. According to an ultra-sonic welding method, a heat may be generated between the components by causing quick, small movements between them. According to one embodiment of the present invention, the heat generated between the components causes the melting/welding of the components.

FIG. 5B depicts the attachment of the rear shell 30 with the front shell 20, in accordance with an embodiment of the present invention. According to one embodiment of the present invention connecting rings 560 and 570, as observed above in respect to FIG. 5A, may be parts of different compartments of device 40. For example, according to one embodiment of the present invention, connecting ring 570 may be part of the rear shell 30 of device 40, while connecting part 560 may be part of the front shell 20. According to some embodiments of the present invention, the rear shell 30 and the front shell 20 may be connected to one another via connecting curve 562 of connecting ring 570 and via convex connecting portion 562 of connecting ring 560.

FIG. 5C is a flow chart diagram 500 of a method for attaching two components in an in-vivo sensing device, for example attachment of the rear shell 30 with the front shell 20, in accordance with an embodiment of the present invention. According to one embodiment of the present invention, the rear shell 30 may be pressed against the front shell (step 592). The concave ring 572 of the rear shell 30 is then latched (step 594) on to the convex connecting portion 562 of the front shell 20 and the two shells 30 and 20 are thus attached (step 596). It should be noted that the connecting positions in the in-vivo sensing components may be reversed as long as concave and convex parts are placed at opposite ends to enable attachment.

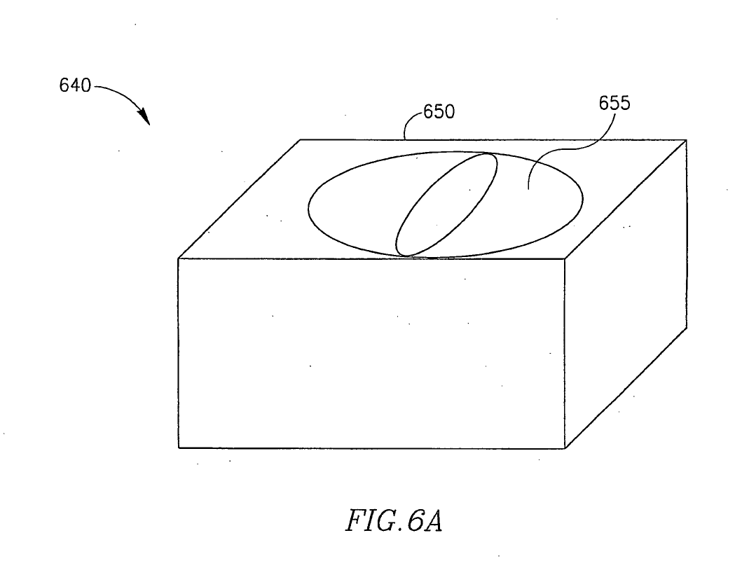

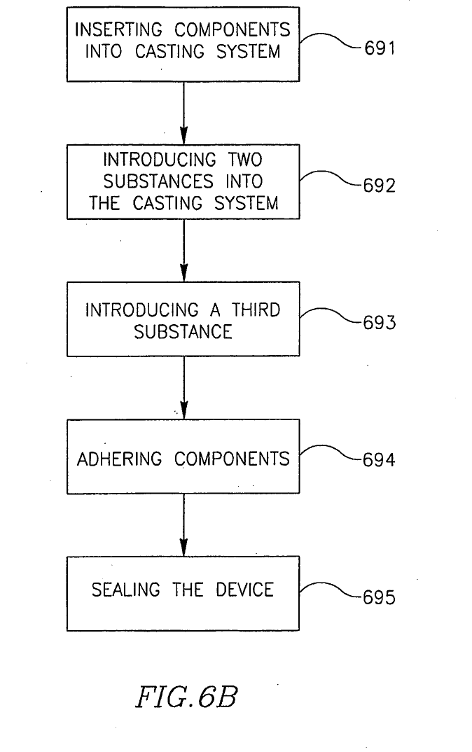

FIG. 6A depicts a casting system 640 for attaching parts of an in-vivo imaging device 40, for example a swallowable capsule, in accordance with an embodiment of the present invention. According to some embodiment of the present invention casting system 640 may include a mold 650, in which, for example a capsule-shaped depression 655 is impressed to it. According to one embodiment of the present invention, the in-vivo device 40 components, such as the electric circuit board, batteries and other components, of an in-vivo imaging device 40 may be inserted into the impression in the mold.

FIG. 6B depicts a method for attaching and sealing an in-vivo sensing device 40 using a mold casting system, in accordance with an embodiment of the present invention. According to one embodiment of the present invention, the components of the in-vivo sensing device 40, for example the imager 146, the illumination source 142, the power source 145, and the circuit board may be inserted, into the casting system 640 (step 691). According to some embodiments of the present invention one or more substances, for example two substances such as a chemical base and a solidifying agent may be introduced into the mold 650 (step 692). According to one embodiment of the present invention, when a third substance, serving as a chemical catalyst for the reaction is introduced (step 693), an ensuing chemical reaction brings about changes in the molecular structure of the materials reacting, and a strong epoxy is formed. The in vivo device 40 components are left until complete solidifying of the material is achieved. According to one embodiment of the present invention the epoxy may be used for adhering the various parts together (step 694). At the end of the attachment process the in vivo sensing components are sealed and all the components are connected as required (step 695).

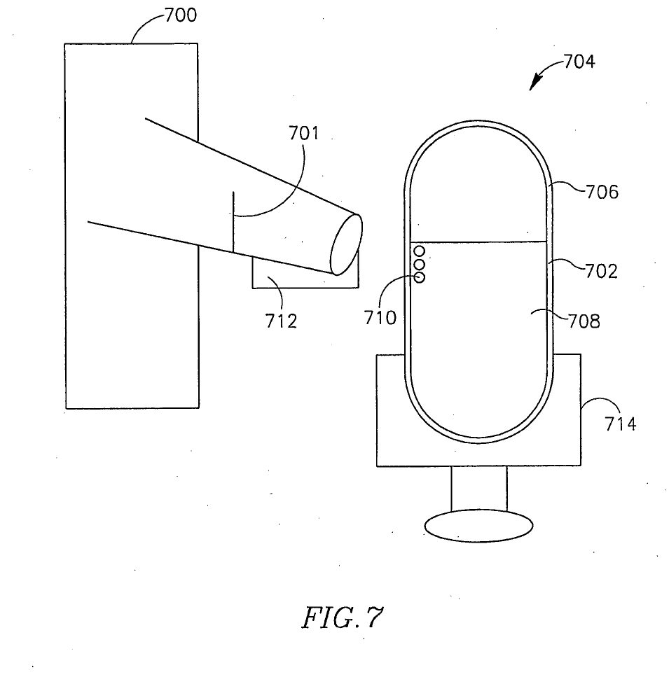

FIG. 7 is a schematic diagram of a laser bonding of thin materials in accordance with an embodiment of the invention. In some embodiments thin materials, such as two or more thin slices or pieces of plastic, resin, polycarbonate, Isoplast or other materials 702 such as for example the shell of a swallowable capsule may be bonded or melted together or sealed with laser energy. In some embodiments a thickness of one or more of the materials to be bonded may be 0.5 mm, though other thicknesses are possible. In some embodiments, two or more of the materials may include segments or shell parts of for example an in vivo sensing device 704 such as for example an in vivo sensor such as a swallowable imager. For example, in some embodiments, a dome 706, such as for example a transparent dome 704 of a swallowable imager 704 may be sealed or bonded to a body 708 of the swallowable imager 704. Other parts of the imager 704, the housing or shell of the imager or of other devices, instruments or components may also be bonded together or sealed with laser energy in accordance with an embodiment of the invention.

In some embodiments, a laser 700 such as for example a DFx03 laser supplied by Rofin of Germany may supply laser energy to seal or bond thin materials. In some embodiments, a pigment 710, such as for example a green pigment as may be supplied by Treffert Company, may be added to one or more of the materials 702 to be bonded or sealed. Laser energy may be applied to an area of the one or more materials that include the pigment 710. The laser energy may melt or energize the pigment 710 in the one or more materials 702, and may bond the two materials 702 together.

In some embodiments laser 700 may be or include a diode laser that may produce laser energy with wavelengths of up to 1064 nanometers (nm). Other spectrums are possible. YAG lasers that may generate laser energy with wavelength in excess of 1064 nm may also be used, such as where pigments 710 in the materials 702 are for example black or dark colored. In some embodiments laser energy may be applied in a continuous mode, though pulse laser energy may be used in some embodiments. In some embodiments, a laser fiber 701 may have a diameter of 600 microns. Other diameters are possible, and other laser sources are possible.

In some embodiments a spot size for laser 700 on a material to be subject to the laser energy may be approximately 600 microns, though underfocusing the laser 700 may create a larger spot size and may avoid burning a material subjected to laser energy. An actual spot size of 900-1000 microns may be possible.

In some embodiments laser 700 may be set to produce 10 watts of laser energy to for example bond one or more materials 702 using a green pigment. Other power levels that may be applied to or suitable for other pigments 710 are possible.

In some embodiments, an absorbtion-spectrum of a pigment 710 that may be included in a thin material 702 to be bonded or sealed by laser 700 may be from 808 nm to 1064 nm. Other absorbtion spectrums may be used.

In some embodiments, a concentration of pigment 710 in the material to be bonded may be or approximate from 6% to 15%. A 10% concentration of pigment in the bonded material 702 is possible. Other concentrations are possible. In some embodiments a pigment 710 may be applied to for example an edge of the material 702 to be bonded, or to another area where bonding is desired. The applied pigment 710 may be subjected to laser energy and may bind a material 702 that may be contiguous to the applied pigment. In some embodiments, a pigment 710 may evaporate or otherwise alter its physical properties when it is subject to laser energy.

In some embodiments, for example when bonding a body 708 of an in vivo sensor 704 to for example a dome 706 such as an optical dome 706 of the sensor 704, a pigment 710 may be included in or applied to such body 708, or to an area of the material 702 that may touch or meet the dome 706. Other application areas are possible. In some embodiments a pigment 710 may be added to or applied to a dome 706, such as for example, in cases where a color of the pigment 710 may not interfere with light collection or other functions of the dome 706.

In some embodiments, a laser 700 may be held in a stationary position and one or more of the materials 702 to be bound or sealed may be rotated on for example a holder 714 or otherwise moved into or out of the path of the laser energy. In some embodiments, a motion of the materials 702 to be bonded, such as for example a material 702 and dome 706 of a swallowable sensor 704 may be moved around for example an axis of the sensor. Other vectors, motions or rotations are possible. Other methods of applying or directing laser energy to a material 702 such as for example by moving a laser beam are possible.

In some embodiments, a burn or application of laser energy may be performed at a location of approximately 0.5 millimeters from the edge of the material 702 that is to be bonded or sealed. In some embodiments, the two or more materials 702 may overlap each other by approximately 1 millimeter. In some embodiments, two or more applications of laser energy are possible as part of a bonding process though other number of applications are possible. In some embodiments, a laser beam or one or more of the materials 702 to which laser energy may be applied, may be moved in for example a zig-zag motion, spiral motion or other non-constant or constant motions, and a bonding of the materials 702 may track such motion.

In some embodiments, parameters of laser energy applied to one or more of the materials 702 may be altered to account for a speed of movement of the material 702 or parts of the material 702 through the energy beam of the laser 700.

In some embodiments a pyrometer 712 may be used in or near the application of laser energy to a thin material and such pyrometer 712 may assist in avoiding burning of the materials 702.

In some embodiments two or more of the materials 702 to be bonded may be touching, and without gaps of air or other materials between them.

The foregoing description of the embodiments of the invention has been presented for the purposes of illustration and description. It is not intended to be exhaustive or to limit the invention to the precise form disclosed. It should be appreciated by persons skilled in the art that many modifications, variations, substitutions, changes, and equivalents are possible in light of the above teaching. It is, therefore, to be understood that the appended claims are intended to cover all such modifications and changes as fall within the true spirit of the invention.

We claim:

1. A shell of a swallowable in vivo sensing device comprising a plurality of shell pieces, said pieces joined together.

2. The shell of claim 1, wherein said pieces comprise a front piece and a back piece.

3. The shell of claim 1, wherein said pieces comprise a front piece, a back piece and a middle piece.

4. The shell of claim 1, wherein said pieces comprise horizontal pieces.

5. The shell of claim 1, comprising a screwing groove.

6. The shell of claim 1, comprising a glue.

7. The shell of claim 1, comprising a connecting ring.

8. The shell of claim 1, comprising a pigment, said pigment to bond said shell pieces upon application of laser energy.

9. The shell of claim 8, wherein said pigment has an absorbtion spectrum of 808 nanometers to 1064 nanometers.

10. The shell of claim 8, wherein a piece of said shell pieces have a concentration of said pigment of between 6% to 15%.

11. The shell of claim 8, wherein said pigment is applied to an edge of a piece of said plurality of shell pieces.

12. A method of assembling a swallowable sensor comprising attaching a first of a plurality of pieces of a shell of said sensor to a second of said plurality of pieces of said shell of said sensor.

13. The method as in claim 12, wherein said attaching comprises a process selected from the group consisting of gluing, welding, screwing, spinning, snapping and applying laser energy.

14. The method as in claim 12, comprising applying laser energy to a pigment in said first piece of said plurality of pieces of said shell of said sensor.

15. The method as in claim 14, comprising rotating said sensor while applying said laser energy.

16. The method as in claim 12, comprising applying a pigment to an edge of a piece of said plurality of pieces, and applying laser energy to said pigment.

17. A method of binding a first material having a thickness of less than 0.5 millimeters to a second material having a thickness of less than 0.5 millimeters, comprising applying laser energy to a pigment in said first material.

18. The method as in claim 17, wherein said applying comprises applying continuous laser energy.

19. The method as in claim 17, wherein said applying comprises applying 10 watts of laser energy.

20. The method as in claim 17, wherein said applying comprises applying laser energy to a spot size of approximately 600 microns.

21. A swallowable capsule comprising a capsule body and a viewing dome, said dome attached to said body by laser welding.

22. The capsule as in claim 21, comprising a pigment that upon the application of laser energy to said pigment, said pigment bonds said body to said dome.
